(19) United States
(12) Patent Application Publication
(10) Pub. No.: US 2005/0054726 A1
(43) Pub. Date: Mar. 10, 2005

(54) Vaccine

(76) Inventor: Lindy Louise Thomsen, Stevenage (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/491,843

(22) PCT Filed: Sep. 26, 2002
(86) PCT No.: PCT/GB02/04365

(30) Foreign Application Priority Data
Oct. 5, 2001 (GB) 0124022.5

(51) Int. Cl. A61K 31/198; A61K 31/275

(52) U.S. Cl. 514/564; 514/527

(57) ABSTRACT

The present invention relates to the use of inducible nitric oxide synthase (iNOS) inhibitors as vaccine adjuvants, and in a preferred aspect of the present invention they are used for adjuvanting nucleic acid vaccines. The present invention further provides pharmaceutical compositions comprising an antigen and the inhibitor.

The present invention relates to the use of inducible nitric oxide synthase (INOS) inhibitors as vaccine adjuvants, and in a preferred aspect of the present invention they are used for adjuvanting nucleic acid vaccines. The present invention further provides pharmaceutical compositions comprising an antigen and the inhibitor.

Nitric oxide (NO) is the endogenous stimulator of the soluble guanylate cyclase enzyme and is involved in a number of biological actions. Excess NO production is also thought to be involved in a number of conditions, including septic shock and many inflammatory diseases. The biochemical synthesis of NO from L-arginine is catalysed by the enzyme NOS. Many inhibitors of NOS have been described and proposed for therapeutic use.

There are currently three types of NOS described, inducible NOS (INOS), neuronal NOS (nNOS), and endothelial NOS (eNOS). More recently, NOS inhibitors displaying selectively for either iNOS, nNOS or eNOS have been disclosed. Selectivity is defined on the basis of relating their potency under identical conditions in the physiological range and can be divided into 3 categories; non-selective, partially selective, and highly selective (W. Alderton, C. Cooper, R. Knowles, “Nitric oxide synthases:Structure, function and inhibition”, In Biochem J. (2001) 357, 593-615). Techniques described in Dawson and Knowles (1998, Methods Mol. Biol., 100, 237-242), measure the concentration of inhibitor (often expressed as μM) required to give a 50% reduction of NO production by the NOS-types in vitro (IC50). Inhibitors with less than 10-fold selectivity for one particular NOS-type (IC50 for 1 NOS-type being <10 fold less than the IC50 of the same inhibitor for another NOS-type) are regarded as non-selective. Inhibitors which have a 10-50-fold selectivity are regarded as partially selective inhibitors, while compounds of over 50-fold selectivity are regarded as highly selective.

Thus WO93/13055 describes selective INOS inhibitors of formula (I) 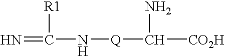
and salts and pharmaceutically acceptable esters and amides thereof, in which:
R₁ is a C₁₋₆ straight or branched chain alkyl group, a C₂₋₆ alkenyl group, a C₂₋₆ alkynyl group, a C₃₋₆cycloalkyl group or a C₃₋₆ cycloalkylC₁₋₆alkyl group;
Q is an alkylene, alkenylene or alkynylene group having 3 to 6 carbon atoms and which may optionally be substituted by one or more C₁₋₃alkyl groups;
a group of formula —(CH₂)ₚX(CH₂)_{q}— where p is 2 or 3, q is 1 or 2 and X is S(O)ₓ where x is 0, 1 or 2, 0 or NR² where R² is H or C₁₋₆alkyl; or
a group of formula —CH₂)ₚX(CH₂)_{q}— where r is 0, 1 or 2, s is 0, 1 or 2 and A is a 3 to 6 membered carbocylic or heterocyclic ring which may optionally be substituted by one or more suitable substituents such as C₁₋₆alkyl, C₁₋₆alkoxy, hydroxy, halo, nitro, cyano, trifluoroC₁₋₆alkyl, amino, C₁₋₆ alkylamino or diC₁₋₆alkylamino.

WO95/34534 discloses iNOS inhibitors which are compounds of formula (II) 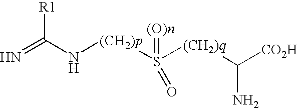
wherein R¹ is a C₁straight or branched chain alkyl group, a C₂₋₆alkenyl group, a C₂₋₆alkynlyl group, a C₃₋₆cycloalkyl group to a C₃₋₆cycloalky C₁₋₆alkyl group, each optionally substituted by one or three groups independently selected from: —CN; —NO₂; a group —COR² wherein R² is hydrogen, C₁₋₆alkyl, —OR³ wherein R³ is hydrogen or C₁₋₆alkyl, or NR⁴R⁵, wherein R⁴ and R⁵ are independently selected from hydrogen or C₁₋₆alkyl; a group S(O)mR₆, wherein m is 0, 1 or 2, R⁶ is hydrogen, C₁₋₆alkyl, hydroxy or NR⁷R⁸, wherein R⁷ and R⁸ are independently hydrogen or C₁alkyl; a group PO(OR⁹)₂, wherein R⁹ is hydrogen or C₁₋₆ alkyl; a group NR¹⁰OR¹¹, wherein R¹⁰ and R¹¹ are independently selected from hydrogen, C₁₋₆ alkyl, —COR¹², wherein R¹² is hydrogen or C₁₋₆alkyl, or —S(O)ₘ,R¹³, wherein m′ is 0, 1 or 2 and R¹³ is hydrogen or C₁₋₆alkyl; halo; or a group-OR⁴, wherein R¹⁴ is hydrogen, C₁₋₆ alkyl optionally substituted by one to three halo atoms, C₆₋₁₀ aryl or —COR¹⁵ wherein R¹⁵ is hydrogen or C₁₋₆alkyl; p is 2 or 3, q is 1 or 2 and no is 0 or 1 and all salts, esters, amides and physiologically acceptable prodrugs thereof.

WO98/30537 discloses compounds falling within the scope of formula I which as well as being selective iNOS inhibitors, display advantages including that they have a long half-life and are orally bioavailable when administered in vivo. In particular those compounds of formula (III) 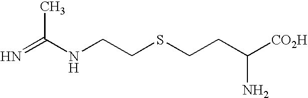
or a salt, solvate, or physiologically functional derivative thereof

NO is known to have a number of roles in the immune system, having both effector and regulatory functions. These functions include direct bacteriocidal effects (K-D Kroncke, K Fehsel, V Kolb-Bachofen. Nitric oxide:Cytotoxicity versus cytoprotection—how, why, when, and where? Nitric Oxide: Biology and Chemistry 1997, 1(2), 107-120), as well as a regulatory role in cytokine expression via caspase activity (Y M Kim, RV Talanian, J L1, T R Billiar, Nitric oxide prevents IL-1beta and IFN-gamma-inducing factor (IL-18) release from macrophages by inhibiting caspase-1 (IL-1beta-converting enzyme). J. Immunology 1998, 161(8), 4122-8), in apoptosis (H T Chung, H O Pae, B M Choi, T R Billier, Y M Kim. Nitric oxide as a bioregulator of apoptosis. Biochemical and Biophysical Research Communications 2001, 282(5), 1075-9), and in adjuvant efficacy for immunotherapies (D A Kahn, D C Archer, D P Gold, C J Kelly, Adjuvant immunotherapy is dependent on inducible nitric oxide synthase. J. Exp. Med. 2001, 193(11), 1261-1267; M M Gherardi, J C Ramirez, M Estaban. Interleukin-12 (IL12) enhancement of the cellular immune response against human immunodeficiency virus type I env antigen in a DNA prime/vaccinia boost vaccine regimen is time and dose dependent: Supressive effects of IL-12 boost are mediated by nitric oxide. J Virology 2000, 74(14), 6278-6286). The present inventors have discovered that NOS inhibitors which inhibit INOS are able to increase immune response to a vaccine antigen. The compositions, methods and uses of the present invention comprise iNOS inhibitors which may be highly selective, partially selective or non-selective iNOS inhibitors.

Accordingly there is provided a method of increasing an immune responses to a vaccine antigen, particularly a cellular immune response comprising administering either sequentially or simultaneously a vaccine antigen and an iNOS inhibitor. Thus there is provided the use of an iNdS inhibitor in the manufacture of a medicament to increase cellular immunity to a vaccine antigen administered simultaneously or sequentially or in combination with the inhibitor.

It will be appreciated that the antigen and the INOS inhibitor may be formulated together in a pharmaceutical composition, and this forms an aspect of the invention. Accordingly there is provided a vaccine composition comprising an iNOS inhibitor and an antigen against which it is desired to generate an immune response.

There is also provided a method of enhancing the immune response against an antigen (DNA or protein or the like) in an individual comprising the administration of the antigen to the individual in association with an INOS inhibitor, either in the form of a combination of the two elements or separate pre- or post-administration of the NOS inhibitor.

The INOS inhibitor may be non-selective, partially selective or a highly selective inhibitor of iNOS in comparison with its activity against the other NOS-types. Preferably the NOS inhibitor is either a partially selective, or a highly selective iNOS inhibitor. Most preferably the RiOS ihibitor used in the present invention is a highly selective iNOS inhibitor.

The “selectivity” of the partially or highly selective iNOS inhibitor used in the present invention is preferably selective over either nNOS or eNOS, and most preferably it is selective over both NNOS and eNOS.

Thus, iNOS inhibitors may be non-selective such as L-NMMA, or partially selective such as L-NIL, or highly selective such as GW274150 (W. Alderton, C. Cooper, K Knowles, “Nitric oxide synthases:Structure, function and inhibition”, In Biochem J. (2001) 357, 593-615).

Inhibitors of INOS that are suitable for use in the vaccines and uses of the present invention typically have an IC₅₀ for INOS of less than 30 μM, and preferably less than 3 M, under defined conditions in vitro (as measured by the techniques described in Dawson and Knowles (supra) and Alderton et al (supra), the contents of which are incorporated herein by reference).

Other INOS inhibitors which may be used in the vaccines of the present invention are described in WO 00/63195, WO 00/44731, WO 00/26195, WO 99/64426, WO 99/46240, WO 99/05131, WO 98/30220, WO 97/32844, WO 97/10204, WO 96/36639, WO 96/35677, WO 96/33175, WO 96/15120, WO 95/25717, WO 95/24382, WO 95/11231, WO 95/11014.

The iNOS inhibitors preferably provide for an increase in antigen specific CD4+ and/or CD8+ T cells. Most preferably iNOS inhibitor containing vaccines of the present invention provide an increase in both CD4+ and CD8+ antigen specific T cell responses. The compounds used in the present invention preferably induce a Th1 biased immune response as measured by the relative increased production of Th1 cytokines, in particular interferon—.

A preferential inducer of a Th1 type of immune response facilitates the generation of a cell mediated response. High levels of Th1-type cytokines tend to favour the induction of cell mediated immune responses to the given antigen, whilst high levels of Th2-type cytokines tend to favour the induction of humoral immune responses to the antigen.

It is important to remember that the distinction of Th1 and Th2-type immune response is not absolute. In reality an individual will support an immune response which is described as being predominantly Th1 or predominantly Th2. However, it is often convenient to consider the families of cytokines in terms of that described in murine CD4 + ve T cell clones by Mosmann and Coffman (Mosmann, T. R. and Coffman, R L. (1989) Th1 and Th2 cells: different patterns of lymphokine secretion lead to different functional properties. Annual Review of Immunology, 7, p145-173). Traditionally, Th1-type responses are associated with the production of the IFN-γ and IL-2 cytokines by T-lymphocytes. Other cytokines often directly associated with the induction of Th1-type immune responses are not produced by T-cells, such as IL-12. In contrast, Th2-type responses are associated with the secretion of IL4, IL-5, IL-6, IL-10. Accordingly, the present invention provides compositions and methods which induce predominantly Th1 type immune responses in the vaccinee.

Preferred compounds for use in the present invention are compounds of formula (I), (II), (III).

Another preferred class of compounds is described in WO 96/19440, formula (IV): 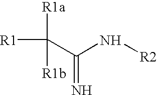
or a salt thereof, wherein, R¹ is hydrogen, a C₁₋₆ hydrocarbyl group optionally substituted by halo, nitro, cyano or a group XR3 wherein X is oxygen, C(O)ₘ wherein m is 1 or 2, S(O)ₙ wherein n is 0, 1 or 2, or a group NR⁴ wherein R⁴ is hydrogen or C₁₋₆alkyl; and R³ is hydrogen, C₁₋₆alkyl, or a group NR⁵R⁶ wherein R⁵ and R⁶ are independently hydrogen or C₁₋₆alkyl, provided that R³ is not NR⁵R⁶ when X is oxygen or S(O)ₙ;
R1a and R1b are independently selected from hydrogen or halo;
R² is a C₁₋₁₄ hydrocarbyl group which may optionally contain one or two heteroatoms, the group R² being optionally substituted by one or more groups independently selected from halo; N₃; nitro, CF₃; ZR⁷ wherein Z is oxygen, C(O)ₘ, wherein m′ is 1 or 2, S(O)ₙ, wherein n′ is 0, 1 or 2, or a group NR⁸ wherein R⁸ is hydrogen or C₁₋₆ alkyl and R⁷ is hrydrogen, C₁₋₆ alkyl or a group NR⁹R¹⁰ wherein R⁹ and R¹⁰ are independently hydrogen or C₁₋₆ alkyl; or R² is substituted by a group 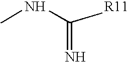
   wherein R¹¹ has a definition the same as for R¹;
   with the proviso that when R¹ is a C₁₋₆alkyl group and R² is a C₁₋₁₄hydrocarbyl substituted by two groups ZR⁷ wherein one group ZR⁷ is CO₂H, the other group ZR⁷ is not NH₂.

Also preferred are specific compounds including a compound of formula (V): 1400W N-[3-(aminomethyl)benzyl] acetamidine), which has the following structure: 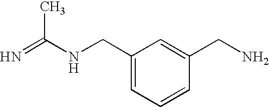
Preferred compounds also include sulphur acetamide substituted amino acids such as: GW 273629 2-(R)-amino-6-(1-imino-ethylamino)-4,4 dioxo-4-thiahexanoic acid, formula (VI), which has the structure: 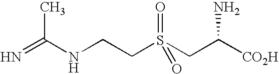
and GW 274150, S-[2-(1-iminoethylamino)ethyl]-L-homocysteine, formula (VII), which has the structure: 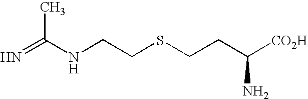
and GW 432042, S-[(R)-2-(1-iminoethylamino)propyl]-L-cysteine, a compound of formula (VIII), which has the structure:

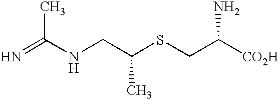

Since, formula (II) includes an asymmetric centre in the amino acid group, and although the natural L or (S) configuration of arginine is preferred, it is intended that formula (I) includes both (S) and (R) enantiomers either in substantially pure form or admixed in any proportions. Likewise, it is envisaged that raecemic mixtures of GW273629, GW274150 and GW432042, or substantially pure (S) and (R) or mixtures thereof may be used in the present invention.

Thus, in the alternative, the present invention provides a compound selected from:

2-(R)-amino-6-(1-imino-ethylamino)4,4 dioxo-4-thiahexanoic acid
2-(S)-amino-6-(1-imino-ethylamino)-4,4 dioxo-4-thiahexanoic acid
2-(RJS)-amino-6-(1-imino-ethylamino)4,4 dioxo-4-thiahexanoic acid
S-[2-(1-iminoethylamino)ethyl]-DL-homocysteine
S-[2-(1-iminoethylamino)ethyl]-L-homocysteine; and
S-[2-(1-iminoethylamino)ethyl]-D-homocysteine
S-[(R)-2-(1-iminoethylamino)propyl]-Lcysteine
S-[(S)-2-(1-iminoethylamino)propyl]-L-cysteine
S-[(R)-2-(1-iminoethylamino)propyl]-D-cysteine
S-[(S)-2-(1-iminoethylamino)propyl]-D-cysteine
S-[(R/S)-2-(1-iminoethylamino)propyl]-D/L-cysteine
S-[(R/S)-2-(1-iminoethylamino)propyl]-L-cysteine
S-[(R/S)-2-(1-iminoethylamino)propyl]-D-cysteine
S-[(R)-2-(1-iminoethylamino)propyl]-D/L-cysteine
S-[(S)-2-(1-iminoethylamino)propyl]-D/L-cysteine
   and salts, solvates, and physiologically functional derivatives thereof.

The iNOS inhibitor for use in the present invention is preferably a compound of formula (I), more preferably a selective inducible NOS inhibitor of formula (II), formula (II), formula (IV), formula (V), formula (VI), formula (VI) or formula (Vw).

Salts and solvates of compounds of formula (I), (II), (III), (V), (V), (VI), (VII) or (VII which are suitable for use as an adjuvant are those wherein the counterion or associated solvent is pharmaceutically acceptable. However, salts and solvates having non-pharmaceutically acceptable counterions or associated solvents are within the scope of the present invention, for example, for use as intermediates in the preparation of other compounds of formula (I), (II), (I), (IV), (V), (VI), (VD) or (VI)) and their pharmaceutically acceptable salts, solvates, and physiologically functional derivatives.

By the term “physiologically functional derivative” is meant a chemical derivative of a compound of formula (I), (II), (III), (IV), (V), (VI), (VII) or (VII) having the same physiological function as the free compound of formula (I), (II), (III, (IV), (V), (VI), (VII) or (VHI) for example, by being convertible in the body thereto. According to the present invention, examples of physiologically functional derivatives include esters, amides and carbamates; preferably esters and amides.

Suitable salts according to the invention include those formed with both organic and inorganic acids or bases. Pharmaceutically acceptable acid addition salts include those formed from hydrochloric, hydrobromic, sulphuric, citric, tartarc, phosphoric, lactic, pyruvic, acetic, trifiuoroaceitc, succinic, oxalic, furmaric, maleic, oxaloacetic, methanesulphonic, ethanesulphonic, ptoluenesulphonic, benzenesulphonic and isethionic acids. Pharmaceutically acceptable base salts include ammonium salts, alkali metal salts such as those of sodium and potassium, alkaline earth metal salts such as those of calcium and magnesium and salts with organic bases such as dicyclohexyl amine and N-methyl-D-glucamine.

Pharmaceutically acceptable esters and amides of the compounds of formula (I) may have the acid group converted to a C₁₋₆alkyl, aryl, aryl C₁₋₆alkyl, or amino acid ester or amide. Pharmaceutically acceptable amides and carbamates of the compounds of formula (I) may have an amino group converted to a C₁₋₆alkyl, aryl, aryl C₁₋₁₆alkyl, or amino acid amide or carbamate.

The vaccines of the present invention may be administered in a conventional liquid form into the tissue of an individual, wherein the INOS inhibitor is formulated with the vaccine antigen, and if present, an additional vaccine adjuvant. However, the vaccine may be provided in the form of a kit in which the vaccine and the INOS inhibitor are administered separately. For example, the vaccine antigen may be administered intramuscularly whilst the INOS inhibitor is administered orally. Alternatively, a ballistic delivery of particulate solid vaccine antigen into the skin may have the iNOS inhibitor associated with the particle or it may be delivered topically at the site of vaccination or delivered orally.

In a particularly preferred embodiment of the present invention the vaccine is delivered into the skin by ballistic delivery and the INOS inhibitor is delivered orally in the form of a tablet In this embodiment the vaccine is preferably a DNA vaccine. Tablet formulation may be readily determined by the man skilled in the art.

The methods of vaccination and treatment of the present invention, therefore, encompass the separate administration of vaccine antigen at one site and the administration of the INOS inhibitor at another site.

The antigen used in the vaccines of the present invention may be a peptide, protein, polysaccharide, protein-polysaccharide conjugate nucleic acid or lipid antigen, but is preferably administered as a nucleic acid, preferably DNA, vaccine for in vivo expression of a protein.

DNA vaccines usually consist of a bacterial plasmid vector into which is inserted a strong viral promoter, the gene of interest which encodes for an antigenic peptide and a polyadenylation/transcriptional termination sequences. The gene of interest may encode a full protein or simply an antigenic peptide sequence relating to the pathogen, tumour or other agent which is intended to be protected against. The plasmid can be grown in bacteria, such as for example E. coli and then isolated and prepared in an appropriate medium, depending upon the intended route of administration, before being administered to the host. Following administration the plasmid is taken up by cells of the host where the encoded peptide is produced. The plasmid vector will preferably be made without an origin of replication which is functional in eukaryotic cells, in order to prevent plasmid replication in the mammalian host and integration within chromosomal DNA of the animal concerned.

There are a number of advantages of DNA vaccination relative to traditional vaccination techniques. First, it is predicted that because the proteins which are encoded by the DNA sequence are synthesised in the host, the structure or conformation of the protein will be similar to the native protein associated with the disease state. It is also likely that DNA vaccination will offer protection against different strains of a virus, by generating cytotoxic T lymphocyte responses that recognise epitopes from conserved proteins. Furthermore, because the plasmids are taken up by the host cells where antigenic protein can be produced, long-lasting cellular and humoral immune responses will be elicited. The technology also offers the possibility of combining diverse immunogens into a single preparation to facilitate simultaneous immunisation in relation to a number of disease states.

Helpful background information in relation to DNA vaccination is provided in Donnelly et al “DNA vaccines” Ann. Rev Immunol. 1997 15: 617-648, the disclosure of which is included herein in its entirety by way of reference.

Despite the numerous advantages associated with DNA vaccination relative to traditional vaccination therapies there is nonetheless a desire to develop improvements which will serve to increase the immune response induced by the protein which is encoded by the plasmid DNA administered to an animal. The present invention addresses these issues. Likewise the present invention can be used with great effect with traditional protein based approaches.

In one embodiment, a composition of the present invention can be delivered via a particle bombardment approach, mrany of which have been described (WO 91/07487). In one illustrative example, gas-driven particle acceleration can be achieved with devices such as those manufactured by Powderject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide, are accelerated to high speed within a helium gas jet generated by a hand held device, propelling the particles into a target tissue of interest, typically the skin. The particles are preferably gold beads of a 0.4-4.0 μm, more preferably 0.6-2.0 μm diameter and the DNA conjugate coated onto these and then encased in a cartridge or cassette for placing into the “gene gun”.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

In the context of protein or DNA vaccination by microparticle ballistic delivery into the skin, the iNOS inhibitors may be applied systemically (such as by direct injection or oral delivery) either prior to, at the same time as, or after vaccination. Alternatively the NOS inhibitors may be applied topically at the site of vaccination before or after the vaccination event.

In an alternate embodiment of the present invention the INOS inhibitor may be formulated with the solid dose delivered particle itself. It is preferred that the vaccine is a DNA vaccine and accordingly the iNOS inhibitor may be formulated with the DNA on gold or tungsten beads, which solid compositions are delivered ballistically into the skin. Accordingly there is provided a solid composition suitable for ballistic delivery into the skin comprising a vaccine antigen and iNOS inhibitor. Preferably the composition comprises a vaccine antigen and an INOS inhibitor (more preferably a partially selective iNOS inhibitor and most preferably a highly selective iNOS inhibitor), and a gold or tungsten bead. Also provided are devices for ballistic delivery of microparticulate vaccines of the present invention into the skin of an individual, comprising the solid compositions described in this paragraph.

The INOS compounds may be administered systemically (orally or via injection) at a dose of from 0.001 to 200 mg/kg per day, preferably 0.01 to 20 mg/kg at or around the time of vaccination. The dose range for adult humans is generally from 0.1 mg to 10 g/day and preferably 1 mg to 1 g/day. Tablets or other forms of presentation provided in discrete units may conveniently contain an amount of compound of the invention which is effective at such dosage or as a multiple of the same, for instance, units containing 0.1 mg to 500 mg, usually around 1 mg to 200 mg.

If the INOS inhibitors are to be formulated for topical administration at the site of injection or for formulation with the vaccine dose or onto the solid vaccine delivery doses the dose of INOS inhibitor may be substantially less than these systemic doses. The suitable doses for these applications can readily be determined by the man skilled in the art. In this context the iNOS inhibitor may be formulated in a topical cream formulation which may be administered by rubbing onto the injection site immediately prior to injection or ballistic delivery onto the site, or may be applied thereafter as appropriately determined by the man skilled in the art.

In a preferred embodiment the antigen is capable of eliciting an immune response against a human pathogen, which antigen or antigenic composition is derived from HIV-1, (such as tat, nef, gp120 or gp160, gp40, p24, gag, env, vif, vpr, vpu, rev), human herpes viruses, such as gH, gL gM gB gC gK gE or gD or derivatives thereof or Immediate Early protein such as ICP27, ICP 47, IC P 4, ICP36 from HSV1 or HSV2, cytomegalovirus, especially Human, (such as gB or derivatives thereof), Epstein Barr virus (such as gp350 or derivatives thereof), Varicella Zoster Virus (such as gpI, II, I and IE63), or from a hepatitis virus such as hepatitis B virus (for example Hepatitis B Surface antigen or Hepatitis core antigen or pol), hepatitis C virus antigen and hepatitis E virus antigen, or from other viral pathogens, such as paramyxoviruses: Respiratory Syncytial virus (such as F and G proteins or derivatives thereof), or antigens from parainfluenza virus, measles virus, mumps virus, human papilloma viruses (for example HPV6, 11, 16, 18, eg L1, L2, E1, E2, E3, E4, E5, E6, E7), flaviviruses (e.g. Yellow Fever Virus, Dengue Virus, Tick-borne encephalitis virus, Japanese Encephalitis Virus) or Iniluenza virus cells, such as HA, NP, NA, or M proteins, or combinations thereof), or antigens derived from bacterial pathogens such as Neisseria spp, including N. gonorrhea and N. meningitidis, eg, transferrin-binding proteins, lactoferrin binding proteins, PilC, adhesins); S. pyogenes (for example M proteins or fragments thereof, C5A protease, S. agalactiae, S. mutans; H. ducreyi; Moraxella spp, including M catarrhalis, also known as Branhamella catarrhalis (for example high and low molecular weight adhesins and invasins); Bordetella spp, including B. pertussis (for example pertactin, pertussis toxin or derivatives thereof, filamenteous hemagglutinin, adenylate cyclase, fimbriae), B. paraperrussis and B. bronchiseptica; Mycobacterium spp., including M. tuberculosis (for example ESAT6, Antigen 85A, -B or C, MPT 44, MPT59, MPT45, HSP10,HSP65, HSP70, HSP 75, HSP90, PPD 19 kDa [Rv3763], PPD 38 kDa [Rvo934]), M. bovis, M leprae, M. avium, M. paratuberculosis, M. smegmatis; Legionella spp, including L. pneumophila; Escherichia spp, including enterotoxic E. coli (for example colonization factors, heat-labile toxin or derivatives thereof, heat-stable toxin or derivatives thereof), enterohemorragic E. coli, enteropathogenic E. coli (for example shiga toxin-like toxin or derivatives thereof); Vibrio spp, including V. cholera (for example cholera toxin or derivatives thereof); Shigella spp, including S. sonnei, S. dysenteriae, S. flexnerii; Yersinia spp, including Y. enterocolitica (for example a Yop protein), Y. pestis, Y. pseudotuberculosis; Campylobacter spp, including C. jejuni (for example toxins, adhesins and invasins) and C. coli; Salmonella spp, including S. typhi, S. paratyphi, S. choleraesuis, S. enteritidis; Listeria spp., including L. monocytogenes; Helicobacter spp, including H. pylori (for example urease, catalase, vacuolating toxin); Pseudomonas spp, including P. aeruginosa; Staphylococcus spp., including S. aureus, S. epidermidis; Enterococcus spp., including E. faecalis, E. faecium; Clostridium spp., including C. tetani (for example tetanus toxin and derivative thereof), C. botulinum (for example botulinum toxin and derivative thereof), C. difficile (for example clostridium toxins A or B and derivatives thereof); Bacillus spp., including B. anthracis (for example botulinum toxin and derivatives thereof; Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the Human Granulocytic Ehrlichiosis; Rickettsia spp, including R. rickettshi; Chlamydia spp., including C. trachomatis (for example MOMP, heparin-binding proteins), C. pneumoniae (for example MOMP, heparin-binding proteins), C. psittaci; Leptospira spp., including L. interrogans; Treponema spp., including T. pallidum (for example the rare outer membrane proteins), T. denticola, T. hyodysenteriae; or derived from parasites such as Plasmodium spp., including P. falciparum; Toxoplasma spp., including T. gondii (for example SAG2, SAG3, Tg34); Entamoeba spp., including E. histolytica; Babesia spp., including B. microti; Trypanosoma spp., including T. cruzi; Giardia spp., including G. lamblia; Leshmania spp., including L. major; Pneumocystis spp., including P. carinii; Trichomonas spp., including T. vaginalis; Schisostoma spp., including S. mansoni, or derived from yeast such as Candida spp., including C. albicans; Cryptococcus spp., including C. neoformans.

Other preferred specific antigens for M. tuberculosis are for example Rv2557, Rv2558, RPFs: Rv0837c, Rv1884c, Rv2389c, Rv2450, Rv1009, aceA (Rv0467), PstS1, (Rv0932), SodA (Rv3846), Rv2031c 16 kDal., Th Ra12, Th H9, Th Ra35, Th38-1, Erd 14, DPV, MTI, MSL, mTTC2 and hTCC1 (WO 99/51748). Proteins for M. tuberculosis also include fusion proteins and variants thereof where at least two, preferably three polypeptides of M tuberculosis are fused into a larger protein. Preferred fusions include Ra12-TbH9-Ra35, Erd14-DPV-MTI, DPV-MTI-MSL, Erd14-DPV-MTI-MSL-mTCC2, Erdl4-DPV-MTI-MSL, DPV-MII-MSL-mTCC2, TbH9-DPV-MTI (WO 99/51748).

Most preferred antigens for Chlamydia include for example the High Molecular Weight Protein (HWMP) (WO 99/17741), ORF3 (EP 366 412), and putative membrane proteins (Pmps). Other Chlamydia antigens of the vaccine formulation can be selected from the group described in WO 99/28475.

Preferred bacterial vaccines comprise antigens derived from Streptococcus spp, including S. pneumoniae (PsaA, PspA, streptolysin, choline-binding proteins) and the protein antigen Pneumolysin (Biochem Biophys Acta, 1989, 67, 1007; Rubins et al., Microbial Pathogenesis, 25, 337-342), and mutant detoxified derivatives thereof (WO 90/06951; WO 99/03884). Other preferred bacterial vaccines comprise antigens derived from Haemophilus spp., including H. influenzae type B (for example PRP and conjugates thereof), non typeable H. influenzae, for example OMP26, high molecular weight adhesins, P5, P6, protein D and lipoprotein D, and fimbrin and fimbrin derived peptides (U.S. Pat. No. 5,843,464) or multiple copy varients or fusion proteins thereof.

The antigens that may be used in the present invention may further comprise antigens derived from parasites that cause Malaria. For example, preferred antigens from Plasmodia falciparum include RTS,S and TRAP. RTS is a hybrid protein comprising substantially all the C-terminal portion of the circumsporozoite (CS) protein of P. falciparum linked via four amino acids of the preS2 portion of Hepatitis B surface antigen to the surface (S) antigen of hepatitis B virus. It's full structure is disclosed in the International Patent Application No. PCT/EP92/02591, published under Number WO 93/10152 claiming priority from UK patent application No.9124390.7. When expressed in yeast RTS is produced as a lipoprotein particle, and when it is co-expressed with the S antigen from HBV it produces a mixed particle known as RTS,S. TRAP antigens are described in the International Patent Application No. PCTIGB89/00895, published under WO 90/01496. A preferred embodiment of the present invention is a Malaria vaccine wherein the antigenic preparation comprises a combination of the RTS, S and TRAP antigens. Other plasmodia antigens that are likely candidates to be components of a multistage Malaria vaccine are P. faciparum MSP1, AMA1, MSP3, EBA, GLURP, RAP1, RAP2, Sequestrin, PIEMP1, Pf332, LSA1, LSA3, STARP, SALSA, PFEXP1, Pfs25, Pfs28, PFS27/25, Pfs16, Pfs48/45, Pfs230 and their analogues in Plasmodium spp.

The invention contemplates the use of an anti-tumour antigen and will be useful for the immunotherapeutic treatment of cancers. For example, tumour rejection antigens such as those for prostate, breast, colorectal, lung, pancreatic, renal or melanoma cancers. Exemplary antigens include MAGE 1, 3 and MAGE 4 or other MAGE antigens such as disclosed in WO99/40188, PRAME, BAGE, Lage (also known as NY Eos 1) SAGE and HAGE (WO 99/53061) or GAGE (Robbins and Kawakami, 1996, Current Opinions in Immunology 8, pps 628-636; Van den Eynde et al., International Journal of Clinical & Laboratory Research (submitted 1997); Correale et al. (1997), Journal of the National Cancer Institute 89, p293. Indeed these antigens are expressed in a wide range of tumour types such as melanoma, lung carcinoma, sarcoma and bladder carcinoma.

MAGE antigens for use in the present invention may be expressed as a fusion protein with an expression enhancer or an Immunological fusion partner. In particular, the Mage protein may be fused to Protein D from Heamophilus influenzae B. In particular, the fusion partner may comprise the first ⅓ of Protein D. Such constructs are disclosed in Wo99/40188. Other examples of fusion proteins that may contain cancer specific epitopes include bcr/abl fusion proteins.

In a preferred embodiment prostate antigens are utilised, such as Prostate specific antigen (PSA), PAP, PSCA (PNAS 95(4) 1735-1740 1998), PSMA or antigen known as Prostase.

Prostase is a prostate-specific serine protease (trypsin-like), 254 amino acid-long, with a conserved serine protease catalytic triad H-D-S and a amino-terminal pre-propeptide sequence, indicating a potential secretory function (P. Nelson, Lu Gan, C. Ferguson, P. Moss, R. Gelinas, L. Hood & K. Wand, “Molecular cloning and characterisation of prostase, an androgen-regulated serine protease with prostate restricted expression, In Proc. Natl. Acad. Sci. USA (1999) 96, 3114-3119). A putative glycosylation site has been described. The predicted structure is very similar to other known serine proteases, showing that the mature polypeptide folds into a single domain. The mature protein is 224 amino acids-long, with one A2 epitope shown to be naturally processed.

Prostase nucleotide sequence and deduced polypeptide sequence and homologs are disclosed in Ferguson, et al. (Proc. Natl. Acad. Sci. USA 1999, 96, 3114-3119) and in International Patent Applications No. WO 98/12302 (and also the corresponding granted patent U.S. Pat. No. 5,955,306), WO 98/20117 (and also the corresponding granted patents U.S. Pat. No. 5,840,871 and U.S. Pat. No. 5,786,148) (prostate-specific kallikrein) and WO 00/04149 (P703P).

The present invention provides antigens comprising prostase protein fusions based on prostase protein and fragments and homologues thereof (“derivatives”). Such derivatives are suitable for use in therapeutic vaccine formulations which are suitable for the treatment of a prostate tumours. Typically the fragment will contain at least 20, preferably 50, more preferably 100 contiguous amino acids as disclosed in the above referenced patent and patent applications.

A further preferred prostate antigen is known as P501 S, sequence D no 113 of WO98/37814. Immunogenic fragments and portions encoded by the gene thereof comprising at least 20, preferably 50, more preferably 100 contiguous amino acids as disclosed in the above referenced patent application, are contemplated. A particular fragment is PS108 (WO 98/50567).

Other prostate specific antigens are known from Wo98/37418, and WO/004149. Another is STEAP PNAS 96 14523 14528 7-12 1999.

Other tumour associated antigens useful in the context of the present invention include: Plu-1 J. Biol. Chem 274 (22) 15633-15645, 1999, HASH-1, HasH-2, Cripto (Salomon et al Bioessays 199, 21 61-70,U.S. Pat. No. 5,654,140) Criptin U.S. Pat. No. 5,981,215. Additionally, antigens particularly relevant for vaccines in the therapy of cancer also comprise tyrosinase and survivin.

The present invention is also useful in combination with breast cancer antigens such as Muc-1, Muc-2, EPCAM, her 2/Neu, mammaglobin (U.S. Pat. No. 5,668,267) or those disclosed in WO/00 52165, WO99/33869, WO99/19479, WO 98/45328. Her 2 neu antigens are disclosed inter alia, in U.S. Pat. No. 5,801,005. Preferably the Her 2 neu comprises the entire extracellular domain (comprising approximately amino acid 1-645) or fragmants thereof and at least an immunogenic portion of or the entire intracellular domain approximately the C terminal 580 amino acids. In particular, the intracellular portion should comprise the phosphorylation domain or fragments thereof. Such constructs are disclosed in WO00/44899. A particularly preferred construct is known as ECDAPD a second is known as ECD PD. (See WO/00/44899.)

The her 2 neu as used herein can be derived from rat, mouse or human.

The vaccine may also contain antigens associated with tumour-support mechanisms (e.g. angiogenesis, tumour invasion) for example tie 2, VEGF.

Vaccines of the present invention may also be used for the prophylaxis or therapy of chronic disorders in addition to allergy, cancer or infectious diseases. Such chronic disorders are diseases such as asthma, atherosclerosis, and Alzheimers and other auto-immune disorders. Vaccines for use as a contraceptive may also be considered.

Antigens relevant for the prophylaxis and the therapy of patients susceptible to or suffering from Alzheimer neurodegenerative disease are, in particular, the N terminal 39-43 amino acid fragment of the (amyloid precursor protein and smaller fragments. This antigen is disclosed in the International Patent Application No. WO 99/27944-(Athena Neurosciences).

Potential self-antigens that could be included as vaccines for auto-immune disorders or as a contraceptive vaccine include: cytokines, hormones, growth factors or extracellular proteins, more preferably a 4-helical cytokine, most preferably IL13. Cytokines include, for example, IL1, IL2, IL3, IL4, IL5, IL6, IL7, IL8, IL9, IL10, IL11, IL12, IL13, IL14, IL15, IL16, IL17, IL18, IL20, IL21, TNF, TGF, GMCSF, MCSF and OSM. 4-helical cytokines include IL2, IL3, IL4, IL5, IL13, GMCSF and MCSF. Hormones include, for example, luteinising hormone (LH), follicle stimulating hormone (FSH), chorionic gonadotropin (CG), VGF, GHrelin, agouti, agouti related protein and neuropeptide Y. Growth factors include, for example, VEGF.

The vaccines of the present invention are particularly suited for the immunotherapeutic treatment of diseases, such as chronic conditions and cancers, but also for the therapy of persistent infections. Accordingly the vaccines of the present invention are particularly suitable for the immunotherapy of infectious diseases, such as Tuberculosis (TB), HIV infections such as AIDS and Hepatitis B (HepB) virus infections.

Accordingly there is provided vaccines comprising the present invention for the immunotherapy of infectious diseases such as TB, AIDS and HepB; and their use in the manufacture of medicaments for the immunotherapy of infectious diseases such as TB, AIDS and HepB. In the context of TB, there is provided a method of treating an individual suffering from TB infection, comprising the administration of a vaccine of the present invention to the individual, thereby reducing the bacterial load of that individual. The reduction of bacterial load, consisting of a reduction of the amount of TB found in the lung sputum, leading to the amelioration or cure of the TB disease.

Also, in the context of AIDS, there is provided a method of treatment of an individual susceptible to or suffering from AIDS. The method comprising the administration of a vaccine of the present invention to the individual, thereby reducing the amount of CD4+ T-cell decline caused by subsequent HIV infection, or slowing or halting the CD4+ T-cell decline in an individual already infected with InV.

Additionally, in the context of persistant Hepatitis B virus infection, there is provided a method of treatment of an individual susceptible to or suffering from HepB infection. Accordingly, there is provided a method comprising the administration of a vaccine of the present invention to the individual, thereby reducing the level of HepB. load in the serum (as measured by DNA clearance) and also reducing the amount of liver damage (as detected by the reduction or stabilisation of serum levels of the enzyme Alanine Transferase (ALT)).

In an embodiment of the invention the antigen is a polynucleotide and is administered/delivered as “naked” DNA, for example as described in Ulmer et al., Science 259:1745-1749, 1993 and reviewed by Cohen, Science 259:1691-1692, 1993. Here the DNA is formulated in a buffered saline solution. The uptake of naked DNA may be increased by coating the DNA onto biodegradable beads, which are efficiently transported into the cells or by using other well known transfection facilitating agents. DNA encoding the antigen may be administered in conjunction with a carrier such as, for example, liposomes. Typically such liposomes are cationic, for example imidazolium derivatives (WO95/14380), guanidine derivatives (WO95/14381), phosphatidyl choline derivatives (WO95/35301), piperazine derivatives (WO95/14651) and biguanide derivatives.

The present invention is exemplified, but not limited to the following examples. Each parameter described in these specific examples may be interpreted as a general feature which is applicable as a preferred aspect of the whole invention.

### EXAMPLES

1. Inducible Nitric Oxide Synthase Inhibitor Increases the Magnitude of the CD4+ T Cell Response to a Nucleic Acid Vaccine

1.1. Construction of Plasmids and DNA Preparation

The plasmids used are based upon pVAC 1, obtained from Michelle Young, GlaxoSmithKline, UK, a modification of the mammalian expression vector, pCI, (Promega), where the multiple cloning site, from EcoRI to Bst ZI, has been replaced by the EMCV IRES sequence flanked 5′ by unique Nhe I, Rsr II and Xho 1 and 3′ by unique Pac I, Asc I and Not I restriction enzyme sites. A chicken ovalbumin expression plasmid, pVAC1.0VA was constructed by ligating PCR amplified cDNA encoding chicken ovalbumin from pUGOVA (a gift from Dr. F. Carbone) into the expression vector pVAC1.

Plasmid DNA was propagated in E. coli, and prepared using plasmid purification kits (QIAGEN Ltd, Crawley, UK), and stored at −20° C. at approximately 1 mg plasmid DNA/ml in 10 mM Tris/EDTA buffer.

1.2. Preparations of Cartridges for Particle Mediated Immunotherapeutic Delivery (PAMI)

Preparation of cartridges for PMID using the Accell gene transfer device was as previously described (Eisenbraun et al DNA and Cell Biology, 1993 Vol 12 No 9 pp 791-797; Pertner et al). Briefly, plasmid DNA was coated onto 2 μm gold particles (DeGussa Corp., South Plainfield, N.J., USA) and loaded into Tefzel tubing, which was subsequently cut into 1.27 cm lengths to serve as cartridges and stored desiccated at 4° C. until use. In a typical vaccination, each cartridge contained 0.5 mg gold coated with 0.5 μg pVAC1.OVA or empty vector (pVAC1).

1.3. Preparation of 1400W for Minipump Delivery In Vivo

1400W was dissolved in sterile water and loaded into minipumps (ALZET Scientific Products, purchased from Charles River, UK). Each minipump provided a continuous infusion of compound at 1.0 μl/hour. Control pumps contained sterile water only.

1.4. Mice and Immunisations

Male and female D0.11.10 transgenic mice (6-10 weeks old) were bred in our specific pathogen-free animal breeding facilities at Bury Green Farm. The transgene that these mice express is the T cell receptor (TCR) specific for a chicken ovalbumin peptide residue (residues 323-339; OVA peptide) bound to MHC-II molecule (I-Ad). The monoclonal antibody, KJ1.26 that specifically recognises this TCR is used for identification of TCR-transgenic T cells.

Balb/c mice were purchased from Charles River United Kingdom Ltd. (Margate, UK).

CD4+ T cell responses were examined using an adoptive transfer model which enhances the sensitivity of the immune parameters to be measured. Here, T cells which specifically recognise a peptide sequence from ovalbumin protein were adoptively transferred from transgenic into naïve wild-type mice before immunisation. Briefly, 24 hours before immunisation, D0.11.10 splenocytes were adoptively transferred into'Balb/c mice at 6-8 weeks of age. For preparation of splenocytes, mice were killed by cervical dislocation and spleens were collected into ice-cold PBS. Splenocytes were teased out into phosphate buffered saline (PBS) followed by lysis of red blood cells (1 minute in buffer consisting of 155 mM NH4Cl, 10 mM KHCO3, 0.1 mM EDTA). After two washes in PBS to remove particulate matter the single cell suspension was adoptively transferred into the lateral tail vein by injection of 100 μl (i.e. 25×106 splenocytes/mouse).

Twenty four hours later, minipumps containing 1400W (delivering 10 mg/kg per hour), or sterile water (controls), were implanted subcutaneously into mice under anaesthesia. Mice were then immediately immunised by PMID with two cartridges of either pVAC1.0VA or empty vector (1 μg plasmid DNA/mouse).

Preparation of CD4 T Cells for Ex Vivo Analyses

Five days later, mice were killed by cervical dislocation and inguinal and periaortic lymph nodes were collected and prepared as for splenocytes (described above), except that the red blood cell lysis step was omitted. To measure the in vivo clonal expansion of CD4+ T cells induced by immunisation, the proportion of ovalbumin-specific T cells was assessed ex vivo in the lymph node cell preparations. Briefly, an aliquot of lymph node cells from each individual mouse was processed for flow cytometry analysis (Coulter XL) using KJ1.26 (0.2 μg, Caltag) and anti-CD4 (0.5 μg, Sigma). The proportion of KJ1+ CD4+ cells was measured within a population with the forward and side scatter of lymphocytes (˜90% of the total lymph node cells). The remaining lymph node cells were pooled within experimental groups, counted and resuspended in medium (RPMI, L-glutamine, penicillin-streptamycin, 2ME) containing 10% FCS for ELISPOT analyses (see example 2, below).

Figure one shows that treatment with pVAC1.OVA+vehicle induces a small increase in clonal expansion compared with empty vector (PVAC1.)+vehicle. The substantial increase in clonal expansion observed with the addition of 1400W exemplifies the adjuvant effect of this compound. No difference was seen between the empty vector+1400W or vehicle group indicating that the effect of 1400W was antigen-restricted.

2. 1400W Induces both Th1 and Th2 Responses to a Nucleic Acid Vaccine.

Th CD4+ subsets were assessed by ELISPOT analyses of CD4+ T cells producing IFN-γ (Th1) and IL-4 (Th2). Briefly, lymph node cell suspension was aliquoted into ELISPOT plates previously coated with capture IFN-γ or IL-4 antibody and stimulated with ovalbumin cognate peptide. After overnight culture, IFN-γ or IL4 producing cells were visualised by application of anti-murine IFN-γ or IL-4 biotin labelled antibody (Pharningen) followed by streptavidin-conjugated alkaline phosphatase and quantitated using image analysis.

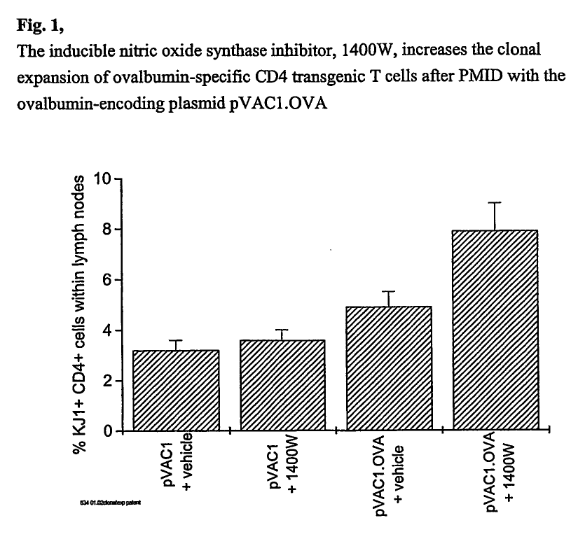

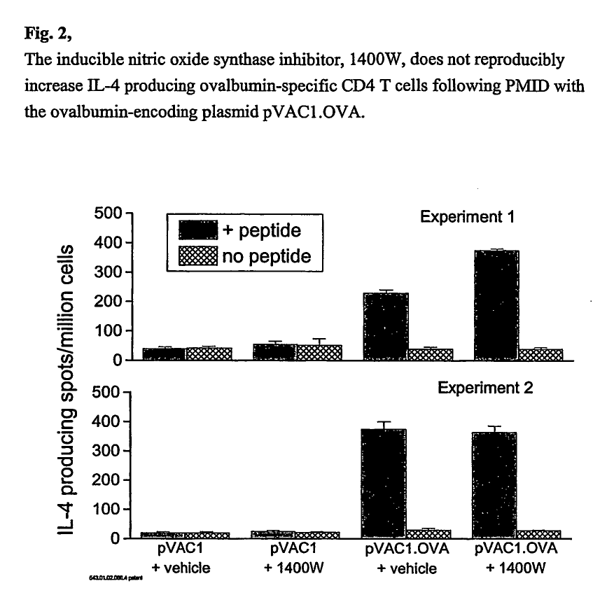

Immunisation with pVAC1.OVA+ vehicle induced an increase in the number of IL-4 producing cells compared with control groups. A variable effect of the combination of 1400W with pVAC1.OVA, ranging from no effect to a modest almost 2 fold increase, was observed (FIG. 2).

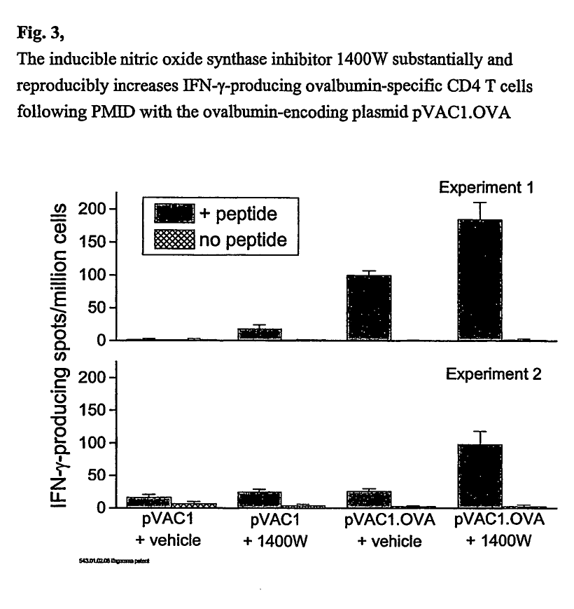

Increases in IFN-γ producing cells were variable for pVAC1.OVA+vehicle compared with control groups (FIG. 3). However, the combination of pVAC1.OVA+1400W substantially and reproducibly increased the number of IFN-γ producing cells by 2-4 times. The bias towards IFN-γ producing cells, indicates that 1400W not only acts as an adjuvant for nucleic acid vaccination but that it preferentially induces a Th1 type of response

3. 1400W Increases the Magnitude of the CD8+ T Cell Response to a Nucleic Acid Vaccine

1.1 Plasmids and Cartridge Preparation

A plasmid expressing cytoplasmically-localised chicken ovalbumin was constructed based on pVAC1 (see example 1) by deletion of an internal Sacd restriction fragment of 378 bp. The deletion is within the region encoding OVA such that the new expressed OVAcyt protein has deleted amino acids 20 to 145 of the OVA protein which include the non-classical secretion signal (Boyle et al., (1997), International Immunology 9: 1897-1906; Tabe et al., (1984), J. Mol. Biol. 180: 645-666). Cartridges were prepared to contain pVAC1.OVAcyt 0.05 μg+pVAC1 0.45 μg (ie. 0.5 μg plasmid DNA/cartridge) as described in example 1. Controls contained pVAC1 (0.5 μg plasmid DNA/cartridge) only.

1.2 Mice and Immunisations

C57B1/6 mice received a primary immunisation followed by a boost immunisation 28 days later, by PMD as described in example 1. Immediately before the boost, mninipumps containing 1400W or sterile water were implanted subcutaneously (see example 1 for methodology). Spleens were collected 12 days later for T cell assays.

1.3 CD8 T Cell Responses

The cytotoxic T cell response was assessed by CD8+ T cell-restricted IFN-γ ELISPOT assay of splenocytes. Mice were killed by cervical dislocation and spleens were collected into ice-cold PBS. Splenocytes were teased out into phophate buffered saline (PBS) followed by lysis of red blood cells (1 minute in buffer consisting of 155 mM NH₄Cl, 10 mM KHCO₃, 0.1 mM EDTA). After two washes in PBS to remove particulate matter the single cell suspension was aliquoted into ELISPOT plates previously coated with capture IFN-γ or IL-2 antibody and stimulated with CD8-restricted cognate peptide. After overnight culture, IFN-γ producing cells were visualised by application of anti-murine IFN-γ-biotin labelled antibody (Pharmingen) followed by streptavidin-conjugated alkaline phosphatase and quantitated using image analysis.

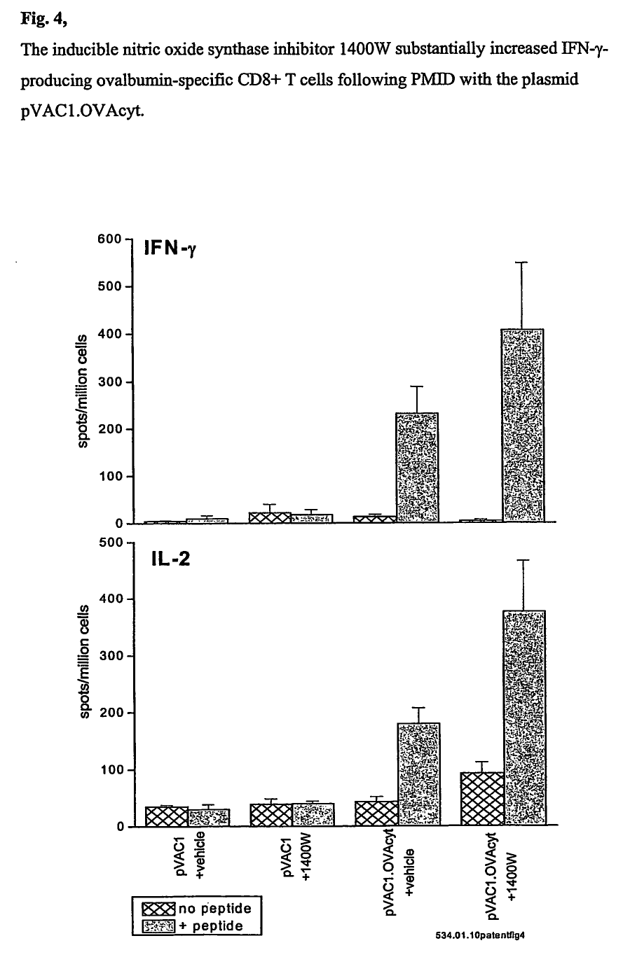

The results of this experiment (FIG. 4) show that the number of IFN-γ- or IL-2-producing CD8+ T cells in the spleens of mice treated with the combination of pVAC1.OVAcyt and 1400W was twice that from mice treated with pVAC1.0VAcyt+vehicle alone. No differences were observed between the control plasmid (PVAC1)+1400W or vehicle groups indicating that the effect of 1400W was antigen-restricted. These results clearly show that 1400W is a potent adjuvant for improving cytotoxic T cell responses following nucleic acid vaccination.

1-10. Cancel

11. A pharmaceutical composition comprising an antigen and an inducible nitric oxide synthase (iNOS) inhibitor.

12. A pharmaceutical composition as claimed in claim 11 comprising an antigen and a compound of formula: 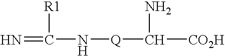
and salts and pharmaceutically acceptable esters and amiders thereof, in which:
R₁ is a C₁₋₆ straight or branched chain alkyl group, a C₂₋₆ alkenyl group, a C₂₋₆ alkynyl group, a C₃₋₆cycloalkyl group or a C₃₋₆ cycloalkylC₁₋₆alkyl group;
Q is an alkylene, alkenylene or alkynylene group having 3 to 6 carbon atoms and which may optionally be substituted by one or more C₁₋₃alkyl groups;
a group of formula —(CH₂)ₚX(CH₂)_{q}— where p is 2 or 3, q is 1 or 2 and X is S(O), where x is 0, 1 or 2, 0 or NR² where R² is H or C₁₋₆alkyl; or
a group of formula —(CH₂)ᵣA(CH₂)ₛ— where r is 0, 1 or 2, s is 0, 1 or 2 and A is a 3 to 6 membered carbocylic or heterocyclic ring which may optionally be substituted by one or more suitable substituents such as C₁₋₆alkyl, C₁₋₆alkoxy, hydroxy, halo, nitro, cyano, trifluoroC₁₋₆alkyl, amino, C₁₋₆ alkylamino or diC₁₋₆alkylamino.

13. A pharmaceutical composition as claimed in claim 12 wherein the compounds are selected from the group: 1400W N-[3-(aminomethyl)benzyl] acetamidine, GW 273629 2-(R)-amino-6-(1-imino-ethylamino)-4,4 dioxo-4thiahexanoic acid, GW 274150, S-[2-(1-iminoethylamino)ethyl]-L-homocysteine, and GW 432042, S-[(R)-2-(1-iminoethylamino)propyl]-L-cysteine.

14. A pharmaceutical composition as claimed in claim 1 wherein the vaccine antigen is selected from the group:
peptides, proteins, polysaccharides, nucleic acid or lipid antigens.

15. A pharmaceutical composition as claimed in claim 1 wherein the antigen is plasmid DNA encoding the antigen.

16. A pharmaceutical composition as claimed in claim 13 wherein the plasmid is coated onto microprojectiles and administered by a ballistic delivery device.

17. A pharmaceutical composition as claimed in claim 14 wherein the microprojectiles are gold beads.

18. A method of increasing an immune response to an antigen comprising administering to a patient in need thereof, the antigen and either sequentialy or simultaneously an inducible nitric oxide synthase (iNOS) inhibitor.

19. The method as claimed in claim 18 comprising an antigen and a compound of formula: 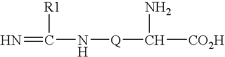
and salts and pharmaceutically acceptable esters and amiders thereof, in which:
R₁ is a C₁₋₆ straight or branched chain alkyl group, a C₂₋₆ alkenyl group, a C₂₋₆ alkynyl group, a C₃₋₆cycloalkyl group or a C₃₋₆ cycloalkylC₁₋₆alkyl group;
Q is an alkylene, alkenylene or alkynylene group having 3 to 6 carbon atoms and which may optionally be substituted by one or more C₁₋₃alkyl groups;
a group of formula—(CH₂)ₚX(CH₂)_{q}— where p is 2 or 3, q is 1 or 2 and X is S(O), where x is 0, 1 or 2, 0 or NR² where R² is H or C₁₋₆alkyl; or
a group of formula —(CH₂)ᵣA(CH₂)ₛ— where r is 0, 1 or 2, s is 0, 1 or 2 and A is a 3 to 6 membered carbocylic or heterocyclic ring which may optionally be substituted by one or more suitable substituents such as C₁₋₆alkyl, C₁₋₆alkoxy, hydroxy, halo, nitro, cyano, trifluoroC₁₋₆alkyl, amino, C₁₋₆ alkylamino or diC₁₋₆alkylamino.

20. The method as claimed in claim 18 wherein the iNOS compounds are selcted from the group: 1400W N-[3-(aminomethyl)benzyl] acetamidine, GW 273629 2-(R)-amino-6-(1-imino-ethylamino)-4,4 dioxo-4-thiahexanoic acid, GW 274150, S-[2-(1-iminoethylamino)ethyl]-L-homocysteine, and GW 432042, S-[(R)-2-(1-iminoethylamino)propyl]-L-cysteine.

21. The method as claimed in claim 18, wherein the vaccine antigen is selected from the group:
peptides, proteins, polysaccharides, nucleic acid or lipid antigens.

22. The method as claimed in claim 18 wherein the antigen is plasmid DNA encoding the antigen.

23. The method as claimed in claim 18 wherein the plasmid is coated onto microprojectiles and administered by a ballistic delivery device.

24. The method as claimed in claim 23 wherein the microprojectiles are gold beads.

25. The method as claimed in claim 18 wherein the immune response is Th1 biased.

26. The method as claimed in claim 18 wherein there is an increase in CD 4+ and/or CD8+ T cells.

27. The method as claimed in claim 18 wherein INOS inhibitor has greater than 50 fold selectivity for inducible nitric oxide synthase.
